(19) United States
(12) Patent Application Publication
(10) Pub. No.: US 2015/0086529 A1
(43) Pub. Date: Mar. 26, 2015

(54) Therapeutic Burn Gel

(71) Applicant: Barbara Ann Hillenbrand, Golden Valley, MN (US)

(72) Inventor: Barbara Ann Hillenbrand, Golden Valley, MN (US)

(21) Appl. No.: 13/975,332

(22) Filed: Sep. 26, 2013

(51) Int. Cl. A61K 38/47; A61K 47/46

(52) U.S. Cl. CPC A61K 38/47; A61K 47/46; C12Y 302/01001
USPC 424/94.61

(57) ABSTRACT

Therapeutic Radiation Burn Gel is an invention that promotes the healing of radiated skin. It works by softening the layers of necrotic tissue which form (in the skin) post radiation. The softening of the necrotic tissue allows for it to be safely and gently peeled away, in order to promote healing. In addition, the Gel is comprised of natural ingredients: Amylase contained in an Aloe Vera gel base. Amylase is an enzyme which is a component of normal human saliva. It softens radiated tissue by way of its lysing action on the sugars and starches which occur naturally in human skin.

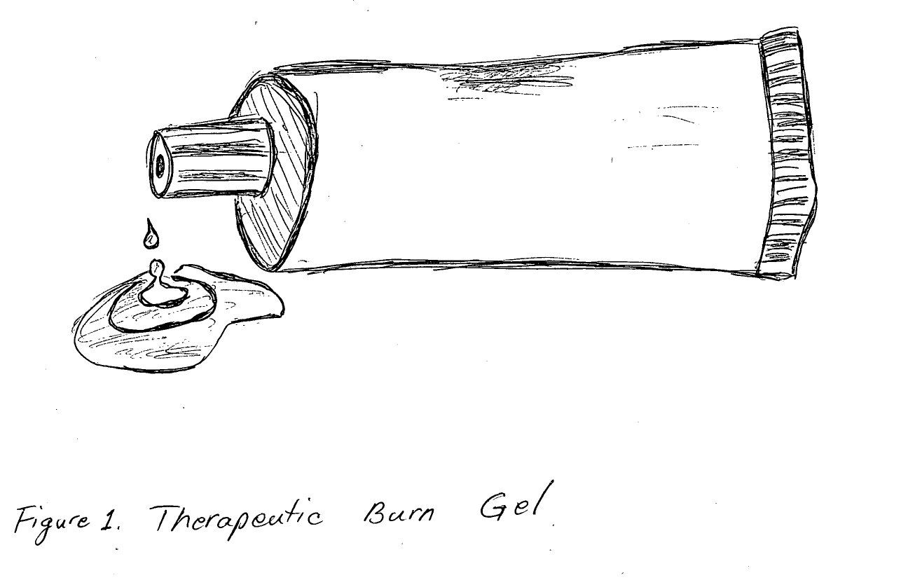

### BACKGROUND AND DESCRIPTION

Radiated and/or severely burned skin often presents, as a hard, slippery, and impenetrable surface, which is difficult to tear away from the underlying layers of epidermis, in order to promote healing. Tearing encrusted tissue away from the healthier tissues below the skin, leads to excessive bleeding and extreme discomfort for the patient. Without an effective method for the removal of radiated and/or burned tissue, proper healing cannot occur, possibly resulting in severe inflammation and/or painful infection below the epidermal surface.

The application of Therapeutic (Radiation) Burn Gel prior to the removal of an encrusted scab, and/or prior to the surgical resection of a radiation wound would result in less bleeding, pain and inflammation at the site after treatment, and, thereby, promote faster healing than would otherwise be possible in the absence of the pretreatment with the Gel.

### BRIEF SUMMARY OF THE INVENTION

Therapeutic Radiation Burn Gel is a combination of a) Amylase (in a concentration which is equivalent to that found in normal human saliva, and at a pH of 7.0); b) and an Aloe-Vera Gel base. In other words, the gel is an admixture of both Amylase and Aloe-Vera. Amylase is an important softening agent has been investigated as a detergent for possible industrial application. For this purposes of the current utility patent application/invention, Amylase (a pre-digestive enzyme) in a Gel form, would be strong enough to soften encrusted radiated tissues, prior to their removal via surgical resection in a Healthcare environment and/or treatment in the patient's home environment. In addition, Aloe-Vera gel has long been used as a therapy for sunburn. Therefore, it is expected that aside from making radiation burns easier to remove, the Gel would also promote healing of the healthier surrounding tissues.

Benefits
Softens hardened scabs and radiated tissue for effective removal.
Features a unique combination of amylase and Aloe-Vera gel.
Promotes healing by exposing healthier tissue lying below the surface of the skin.
Prevents destruction of healthy tissue surrounding the wound.
Results the amount of bleeding that is associated with the surgical or manual resection of scabs and/or radiated tissue.

### DRAWING ATTACHED

See drawing in attached image file. The image is of a tube containing the Therapeutic Radiation Burn Gel. Each tube would contain 4 to 6 ounces of the Gel product.

### DETAILED DESCRIPTION OF THE INVENTION

Therapeutic Burn Gel provides a safe and effective way to soften hardened, encrusted tissue for quick and easy removal. This innovative product may comprise a mixture of Aloe-Vera gel and concentrated Amylase (in its biologically active form at a pH of 7.0 and at a concentration equivalent to that of normal human saliva). The mixture could be packaged in either a 4-ounce or a 6-ounce tube. An extra strength formulation containing a double concentration of Amylase may also be offered (pending the safety findings of clinical investigators). The gel can be used as an adjunct tool for routine surgical resection within a hospital, and/or outpatient environment. Additionally, the Gel could be used at home for the therapeutic relief of less severe radiation or burn wounds. The gel should be applied to the scab or damaged tissue, 1 to 2 hours prior to daily wound care. The exact specifications may vary upon manufacturing.

1. Therapeutic Radiation Burn Gel promotes healing by softening scabs that result From radiation to the skin.

2. The softening effect of Therapeutic Radiation Burn Gel is due primarily to the lysing action of Amylase on sugars and starches that occur naturally in the skin.
